(19) United States
(12) Patent Application Publication
(10) Pub. No.: US 2019/0231579 A1
(43) Pub. Date: Aug. 1, 2019

(54) WRINKLE PREVENTING DEVICE

(71) Applicant: Dana Angelica Forbes, Naples, FL (US)

(72) Inventor: Dana Angelica Forbes, Naples, FL (US)

(21) Appl. No.: 15/884,685

(22) Filed: Jan. 31, 2018

(51) Int. Cl. A61F 5/37; A61F 9/04; A41D 20/00; A41D 27/08

(52) U.S. Cl. CPC A61F 5/3707; A41D 27/08; A41D 20/00; A61F 9/04

(57) ABSTRACT

A wrinkle prevention device is provided. The wrinkle prevention device includes a headband providing mini-pillows that can be selectively placed along the headband so as to minimize their bulk when preventing wrinkles during a restful sleep. The mini pillows are dimensioned and adapted to lift the side of a user's face off their preferred pillow. The headband may have an elastic portion for both providing a comfortable fit along as a non-slip surface preventing the mini pillows for moving during sleep.

### BACKGROUND OF THE INVENTION

The present invention relates to wrinkle prevention devices and, more particularly, to a device for preventing wrinkles that embodies a headband providing mini-pillows that can be selectively placed along the headband so as to minimize the bulk when preventing wrinkles during a restful sleep, while affording many other advantages over the current solutions to pillow-induced wrinkles.

Nobody likes wrinkles, and many people try mightily to prevent them. Most people, however, do not know that there are two types of wrinkles: a first wrinkle type caused by loss of collagen in the skin naturally through the aging process; and a second wrinkle type caused by sleeping on one's face, either when sleeping on our side or face down. Thus, a good night's sleep may leave us refreshed, but with disturbing lines and wrinkles across our face from the pillow hugging our face during sleep.

Current solutions employ “beauty pillows” having substantial volume that covers a large portion of the user's head, making the user feel hot during the sleep, and sometime uncomfortable. Moreover, the bulk of current beauty pillow afford limited sleep area, forcing the user to sleep in the same position as they cannot move freely nor turn around when employing such large pillows. Accordingly, current beauty pillows are difficult to take along on trips as their bulk takes up valuable space during transport. Additionally, because of the bigness of current beauty pillows, a user needs to stop using their preferred pillow, which can adversely affect their sleep.

Furthermore, these current devices do not protect the user's ear from being crushed on the pillow under the weight of the head. Likewise, they are not supporting the user's head against flopping to the side when the user sleeps on their back.

The second type of sleep wrinkle can still be prevented just by wearing much smaller pillows that prevent the wrinkle-inducing pillow interface formed by compressing the face against the pillow, but with none of the abovementioned disadvantages. Unfortunately, nothing currently exists that takes advantage of this inventive concept.

As can be seen, there is a need for the present invention providing two mini pillows provided along opposing sides of headgear so as to keep a user's face spaced apart from their preferred pillow. The device of the present invention utilizes a headband structure supporting the two mini-pillows that cover only a small portion of the user's head, and can that be selectively located along the user's temple keeping a user's head much cooler during the night compared to current beauty pillows. The selective placement of the two mini-pillows along the headgear keeps the user's ears safe for mashing into the pillows, while supporting their head and preventing the head from flopping to the side if they are sleeping on their back, avoiding morning neck pain. The compact nature of the present invention also makes traveling much easier than current beauty pillows.

### SUMMARY OF THE INVENTION

In one aspect of the present invention, a wrinkle prevention device includes a headband adapted to be worn along a forehead of a human wearer; and at least one pillow attached to the headband, each pillow dimensioned to be located adjacent to a temple portion and an ear portion to be located immediately above an ear of the human wearer when the headband is worn.

In another aspect of the present invention, the above wrinkle prevention device wherein each pillow provides a removable fastener for selectively locating each pillow along the headband.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, shown in use;

FIG. 2 is a perspective view of an exemplary embodiment of the present invention;

FIG. 3 is a perspective view of an exemplary embodiment of the present invention;

FIG. 4 is an exploded perspective view of an exemplary embodiment of the present invention;

FIG. 5 is a cross-sectional view of an exemplary embodiment of the present invention, taken along line 5-5 in FIG. 3; and

FIG. 6 is a perspective view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a headband providing mini-pillows that can be selectively placed along the headband so as to minimize their bulk when preventing wrinkles during a restful sleep. The mini pillows are dimensioned and adapted to lift the side of a user's face off their preferred pillow. The headband may have an elastic portion for both providing a comfortable fit along as a non-slip surface preventing the mini pillows for moving during sleep.

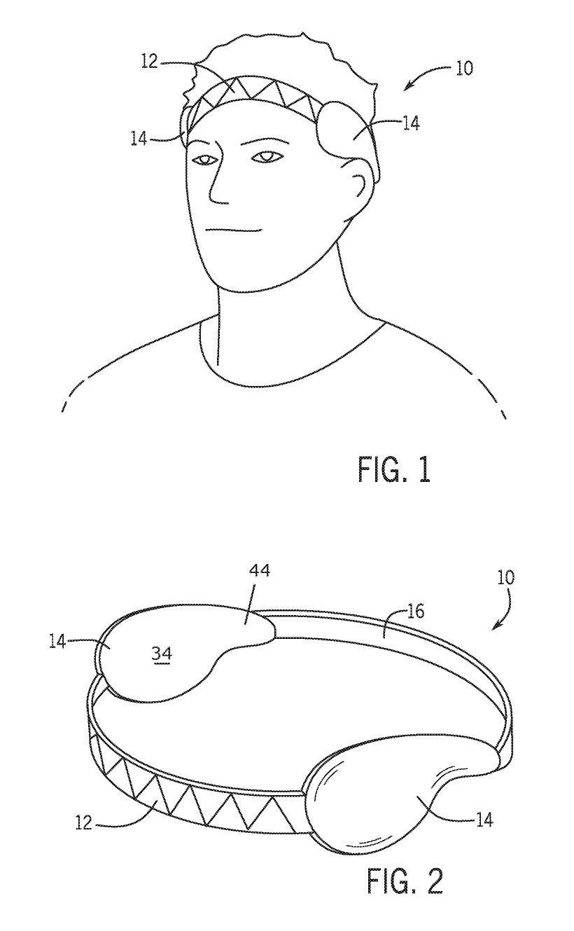

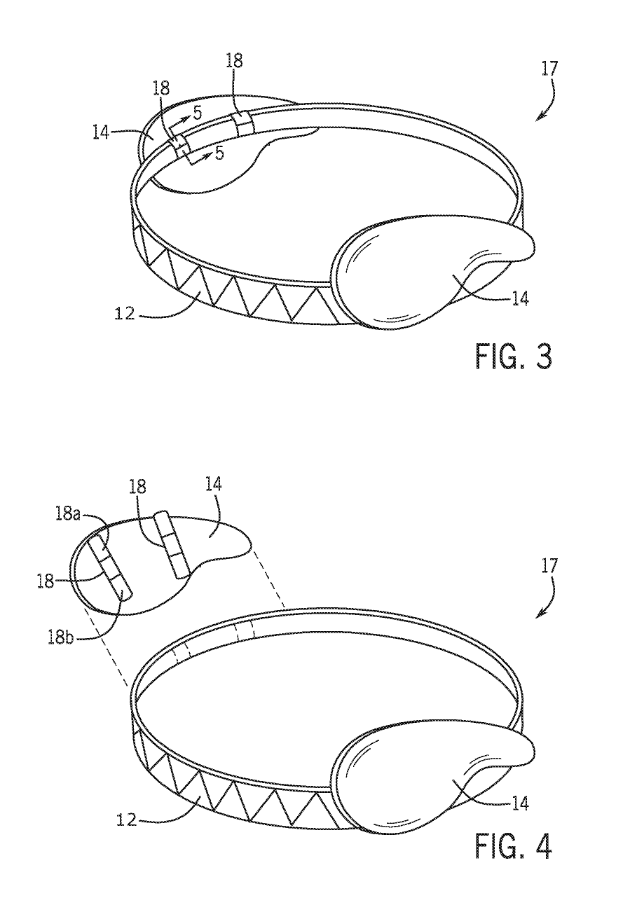

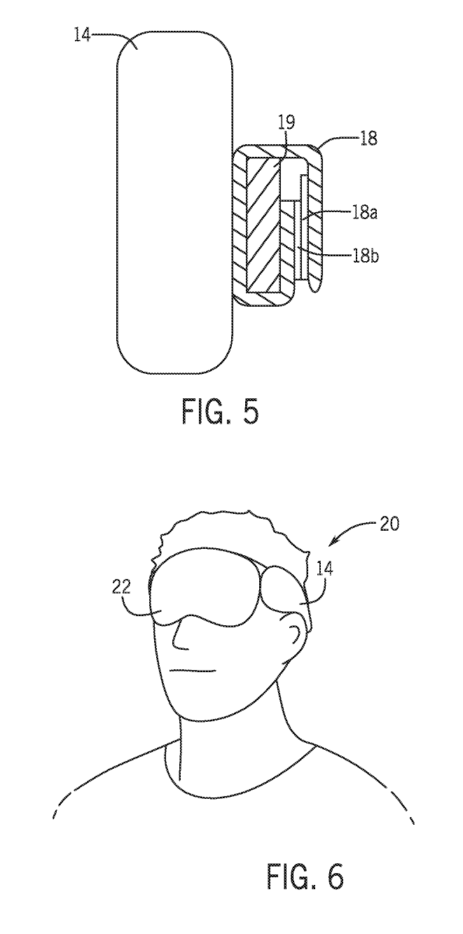

Referring to FIGS. 1 through 6, the present invention may include a wrinkle prevention device 10 providing a headband 12 and at least one pillow 14 that can be selectively attached along the headband 12. Each pillow 14 may be teardrop or paisley shaped. Each pillow 14 may be dimensioned and adapted to be located against the temple of a wearer of the headband 12. Each pillow 14 has a first portion 34 and a curved tapered second portion 44 adapted to allow an upper earlobe to nest against the second portion 44, while the first portion 34 covers the temple of the wearer of the wrinkle prevention device 10. The first portion 34 may be generally oval and have a major axis (think ellipse) approximately three to five inches in diameter. The first portion 34 may have a thickness of approximately one inch.

The headband 12 may provide a segment of elastic material 16 adapted to most likely wrap around the back portion of the head of a wearer of the wrinkle prevention device 10, providing stretch to fit many differently sized heads.

In a second embodiment 17 each pillow 14 may be separable from the headband 12 by providing an adjustable fastener 18 having male and female components 18a and 18b for making them removable, and thus making the placement of each pillow 14 selective along the length of the headband 12. In certain embodiments, the male and female components 18a and 18b may be hook and loop portions of a hook and loop fastener at the opposing ends of a strap. In a third embodiment 20, the headband 12 may provide an eye mask 22, as illustrated in FIG. 6.

A method of using the present invention may include the following. The wrinkle prevention device 10 disclosed above may be provided. A user may don the headband 12 as illustrated in FIG. 1 so that each pillow 14 is located adjacent to and covering the wearer's temple and extending behind the wearer's ears, preventing morning ear ache and wax deafness. When sleeping on their side, the first portion 34 will lift up the head from the user's preferred bed pillow (not shown) just enough to keep the user's face away from the pressure causes the formation of wrinkle. The pillows 14 will also keep the ear free of pressure, and if sleeping on the back the mini pillows 14 will support the user's head from flopping to the side, which can cause neck pain. The selectively placed pillows 14 can be adjusted to comfort. The elastic 16 in the back can be a non-slip elastic to prevent sliding of the pillows 14 during sleep.

Because the pillows 14 are located over the ears yet cover only a small portion of the head—from temple to the end of the ear—and by having only the headband 12 over the forehead and a simple elastic 16 in the back, the present invention is much lighter than current solutions.

It should be understood, of course, that the foregoing relates to exemplary embodiments of the invention and that modifications may be made without departing from the spirit and scope of the invention as set forth in the following claims.

What is claimed is:

1. A wrinkle prevention device, comprising:
a headband adapted to be worn along a forehead of a human wearer; and
at least one pillow attached to the headband, each pillow dimensioned to be located adjacent to a temple portion and an ear portion to be located immediately above an ear of the human wearer when the headband is worn.

2. The wrinkle prevention device of claim 1, wherein each pillow is paisley shaped.

3. The wrinkle prevention device of claim 2, wherein the paisley shaped includes a first portion to cover the temple portion and a second portion to cover the ear portion.

4. The wrinkle prevention device of claim 3, wherein the first portion 34 is generally oval having a major axis between three to five inches in diameter.

5. The wrinkle prevention device of claim 4, wherein the first portion has a thickness of approximately one inch.

6. The wrinkle prevention device of claim 5, wherein each pillow provides a removable fastener for selectively locating each pillow along the headband.

7. The wrinkle prevention device of claim 6, the headband including an elastic portion.

8. The wrinkle prevention device of claim 7, the elastic portion providing non-slip properties to prevent the at least one pillow from slipping from its selected location.

9. The wrinkle prevention device of claim 8, further comprising a eye mask attached to the headband.

10. The wrinkle prevention device of claim 1, the headband including an elastic portion.

11. The wrinkle prevention device of claim 10, the elastic portion providing non-slip properties to prevent the at least one pillow from slipping from its selected location.

12. The wrinkle prevention device of claim 11, further comprising a eye mask attached to the headband.
